(19) United States
(12) Patent Application Publication
(10) Pub. No.: US 2009/0023213 A1
(43) Pub. Date: Jan. 22, 2009

(54) Method to increase the rate of transgenic embryo formation after inoculation of immature cotyledons with agrobacterium

(75) Inventors: Marina Sigareva, Chapel Hill, NC (US); Geeta Menon, Scott, MS (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Syngenta Participations AG, Basel (CH)

(21) Appl. No.: 11/880,110

(22) Filed: Jul. 19, 2007

(51) Int. Cl. A01H 5/00; C12N 5/04

(52) U.S. Cl. 435/415; 800/312

(57) ABSTRACT

The present disclosure provides methods for the transformation of soybean cells or tissue and regeneration of the soybean cells or tissue into transformed plants. The disclosed methods utilize an explant prepared from an immature soybean cotyledon which can be induced directly to form shoots that give rise to transgenic plants via embryogenesis.

### TECHNICAL FIELD

The presently disclosed subject matter relates generally to methods for plant transformation and, more particularly, to methods for transforming soybean cells or tissues by embryogenesis. The presently disclosed subject matter also relates to methods for regenerating transgenic soybean plants from transformed soybean cells or tissues. The presently disclosed subject matter further relates to transgenic soybean plants and seeds obtained by such methods.

TABLE OF ABBREVIATIONS

| | |
|---|---|
| 2,4-D | 2,4-dichlorophenoxyacetic acid |
| CMV | Cauliflower Mosaic Virus |
| DNA | deoxyribonucleic acid |
| g | gram |
| GFP | Green fluorescent protein |
| h | hour |
| IAA | indole-3-acetic acid |
| IBA | 3-indolebutyric acid |
| ID | identification |
| L | liter |
| mg | milligram |
| min | minute |
| ml | milliliter |
| mm | millimeter |
| MS | Murashige & Skoog |
| OD | optical density |
| sec | second |
| TF | transformation frequency |
| ° C. | degrees Celsius |
| % | percent |
| > | greater than |
| < | less than |
| ≧ | greater than or equal to |
| ≦ | less than or equal to |

### BACKGROUND

Cultivated soybean (Glycine max) is a major food and feed crop, with a substantial commercial value throughout the world. Over 50 million hectares worldwide are used to produce an annual crop of soybeans in excess of 100 metric tons with an estimated value exceeding 20 billion dollars. Unfortunately, only a few plant introductions have given rise to the major cultivars grown in the United States and, as a consequence, the narrow germplasm base has limited soybean breeding potential. The limited genetic base in domestic soybean varieties has limited the power of traditional breeding methods to develop varieties with improved or value-added traits. The development of scientific methods useful in improving the quantity and quality of this crop is therefore of significant commercial interest.

Modern biotechnological research and development have provided useful techniques for the improvement of agricultural products by plant genetic engineering. Plant genetic engineering involves the transfer of a desired gene or genes into the inheritable germline of crop plants such that those genes can be bred into or among the elite varieties used in modern agriculture. Gene transfer techniques allow the development of new classes of elite crop varieties with improved disease resistance, herbicide tolerance, and increased nutritional value. Various methods have been developed for transferring genes into plant tissues, including high velocity microprojection, microinjection, electroporation, direct DNA uptake, and Agrobacterium-mediated gene transformation.

Transformation systems employing the bacterium Agrobacterium tumefaciens have conventionally been used for the genetic transformation of soybean plants. In addition, high velocity microprojectile bombardment has also been used as an alternative method for the genetic transformation of soybean plants.

Although advances have been made in the field of plant transformation, a need continues to exist for improved methods to facilitate the ease, speed, and efficiency of such methods for the transformation of soybean plants.

### SUMMARY

The presently disclosed subject matter provides a method for transforming soybean cells or tissues via embryogenesis. In some embodiments, the methods comprise preparing an explant from an immature soybean cotyledon, contacting the explant with a genetic construct, and culturing the explant in the presence of a selection agent.

In some embodiments, preparing the explant comprises one or more of the following: (a) isolating seeds from seed pods and sterilizing the seeds; (b) removing the seed coat; (c) isolating the immature cotyledons from the explant; (d) pre-culturing the immature cotyledons; and (e) wounding the explant.

In some embodiments, wounding the explant comprises one or more of the following: (i) contacting a mechanical instrument with the abaxial or adaxial sides of the cotyledons; and (ii) bombarding the explant tissue with a microprojectile on the abaxial or adaxial sides of the cotyledons.

In some embodiments, the genetic construct comprises a gene of interest and/or a selectable marker gene.

In some embodiments, the selectable marker gene confers antibiotic or herbicide resistance to the explant.

In some embodiments, the antibiotic is selected from the group consisting of: cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, vancomycin, and combinations thereof.

In some embodiments, the herbicide is selected from the group consisting of: glyphosate, sulfonylurea, imidazolinone, glufosinate, bialophos, phenoxy proprionic acid, cycloshexone, triazine, benzonitrile, HPPD inhibitors and combinations thereof.

In some embodiments, the contacting comprises contacting the explant with an Agrobacterium cell comprising the genetic construct.

In some embodiments, the Agrobacterium is Agrobacterium tumefaciens.

In some embodiments, the explant is contacted with the Agrobacterium for about 45 minutes.

In some embodiments, the explants are further co-cultured with Agrobacterium for up to about 5 days.

In some embodiments, the contacting comprises delivering the genetic construct to the explant using a ballistic device.

In some embodiments, the explant can be further cultured in a medium comprising a plant hormone.

In some embodiments, the pre-culturing is for up to about 5 days.

In some embodiments, the contacting comprises abrading, piercing, poking, penetrating with fine particles or pressurized fluids, plasma wounding, applying of hyperbaric pressure, sonicating, or combinations thereof.

In some embodiments, the mechanical instrument comprises a scalpel, pair of scissors, needle, abrasive object, airbrush, particle, vacuum infiltration, electric gene gun, sound wave, or combinations thereof.

In some embodiments, the microprojectile comprises metal, glass, silica, ice, polyethylene, polypropylene, polycarbonate, carbon compounds, and combinations thereof.

In some embodiments, the metal is gold.

In some embodiments, the presently disclosed subject matter comprises transgenic soybean cells or tissues.

In some embodiments, the presently disclosed subject matter comprises soybean plants regenerated from transgenic soybean cells or tissues.

In some embodiments, the presently disclosed subject matter comprises transgenic seeds produced by the disclosed transgenic soybean plants.

Accordingly, it is an object of the presently disclosed subject matter to provide for the transformation of immature soybean cotyledons through embryogenesis.

An object of the presently disclosed subject matter having been stated hereinabove, and which is achieved in whole or in part by the presently disclosed subject matter, other objects will become evident as the description proceeds.

### DETAILED DESCRIPTION

### I. General Considerations

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

Currently in the art, transforming plants from immature embryos can be achieved using an embryogenesis approach. See, for example, U.S. Pat. Nos. 6,858,777 and 5,569,834. Embryogenic transformation is typically coupled with biolistic gene delivery methods with proliferating embryogenic calluses being the target for transformation. At least one disadvantage of the biolistic gene delivery method is the potential for high copy numbers and the fragmentation of transgenes.

An alternative method of gene delivery for plant transformation currently available in the art is Agrobacterium-mediated gene delivery. Agrobacterium-mediated gene delivery can result in reduced copy numbers and increased intact insertions of transgenes compared to biolistic methods. However, employing traditional methods of Agrobacterium transformation of immature zygotic cotyledons coupled with embryogenesis can provide substantially reduced transformation frequencies.

Organogenic regeneration of transformed plants, usually coupled with Agrobacterium-mediated gene delivery, is an alternative to embryogenesis. However, one disadvantage of this method is the potential to produce a high percentage (up to ˜50%) of chimeric plants, wherein the plants do not transmit the transgenes to the progeny.

Provided in the presently disclosed subject matter are embryogenic methods of transforming plants comprising an Agrobacterium-mediated gene delivery system using cotyledons from immature embryos, wherein the transgenic plants are generated with a notably higher transformation frequency and substantially reduced chimerism in the progeny as compared to the currently available embryogenic and organogenic methods.

### II. Definitions

Unless otherwise defined, all technical and scientific terms used herein have the same meaning as commonly understood by one of ordinary skill in the art to which the presently disclosed subject matter pertains. For clarity of the present specification, certain definitions are presented herein below.

Following long-standing patent law convention, the terms “a” and “an” mean “one or more” when used in the subject application, including the claims.

As used herein, the term “about”, when referring to a value or to an amount of mass, weight, time, volume, concentration or percentage is meant to encompass variations of ±20% or ±10%, in another example ±5%, in another example ±1%, and in still another example ±0.1% from the specified amount, as such variations are appropriate to practice the presently disclosed subject matter. Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “adaxial” refers to the side of a plant situated toward the axis or central line.

As used herein, the term “abaxial” refers to the side of a plant situated away from the axis or central line.

As used herein, the term “callus” refers to the actively dividing non-organized masses of undifferentiated and differentiated cells and/or tissue that develop on or around an wounded or cut plant surface, or that develop during tissue culture of plant parts.

As used herein, the term “cotyledon” refers to the first, first pair, or first whorl of leaf-like structures on a plant embryo that function primarily to make food compounds in the seed available to the developing totipotent plant tissue.

As used herein, the term “DNA segment” refers to a DNA molecule that has been isolated free of total genomic DNA of a particular species. Included within the term “DNA segment” are DNA segments, smaller fragments of such segments, and recombinant vectors, including but not limited to plasmids, cosmids, phages, viruses, and the like.

As used herein, the phrase “enhancer-promoter” refers to a composite unit that contains both enhancer and promoter elements. An enhancer-promoter is operatively linked to a coding sequence that encodes at least one gene product.

As used herein, the term “embryo axis” refers to the embryo organs or parts, including the plumule, epicotyl, cotyledonary node, hypocotyl, and radicle. In some embodiments, the term “embryo axis” refers to the longitudinal central line around which the organs or parts of the embryo are arranged.

As used herein, the term “expression cassette” refers to a nucleic acid molecule capable of directing expression of a particular nucleotide sequence in an appropriate host cell, comprising a promoter operatively linked to the nucleotide sequence of interest which is operatively linked to termination signals. It also can comprise sequences required for proper translation of the nucleotide sequence. The coding region can encode a polypeptide of interest and can also encode a functional RNA of interest, including but not limited to, antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression. In some embodiments, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular tissue, organ, or stage of development.

As used herein, the term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” as used herein refers to the cellular processes by which a biologically active polypeptide is produced from a DNA sequence.

The term “germination” as used herein refers to the process whereby growth emerges in a seed from a period of dormancy. Germination typically involves the proper levels of water, oxygen, temperature, and the proper supporting media to begin growth.

As used herein, the terms “heterologous”, “recombinant”, and “exogenous”, when used herein to refer to a nucleic acid sequence (e.g., a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through methods including, but not limited to, the use of DNA shuffling or other recombinant techniques (such as but not limited to cloning the gene into a vector). The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

Accordingly, a polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

The term “pod” as used herein refers to the fruit of a soybean plant. It includes the hull or shell (pericarp) and the soybean seeds.

The term “promoter region” defines a nucleotide sequence within a gene that is positioned 5′ to a coding sequence of a same gene and functions to direct transcription of the coding sequence. The promoter region includes a transcriptional start site and at least one cis-regulatory element. A “functional portion” of a promoter gene fragment is a nucleotide sequence within a promoter region that is required for normal gene transcription. To determine nucleotide sequences that are functional, the expression of a reporter gene is assayed when variably placed under the direction of a promoter region fragment.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operably linked to a transcriptional regulatory region can be found in Alam & Cook (1990) Anal Biochem 188:245-254 and PCT International Publication No. WO 97/47763. Non-limiting examples of reporter genes suitable for transcriptional analyses include the lacZ gene (See, e.g., Rose & Botstein (1983) Meth Enzymol 101:167-180), Green Fluorescent Protein (GFP) (Cubitt et al. (1995) Trends Biochem Sci 20:448-455), luciferase, or chloramphenicol acetyl transferase (CAT). Any suitable reporter and detection method can be used in accordance with the presently disclosed methods, and it can be appreciated by one of skill in the art that no particular choice is essential to or a limitation of the presently disclosed subject matter.

“Shoot” as used herein refers to the aerial portions of the plant and includes the stem, leaves, axillary meristems and apical meristem.

As used herein, the term “totipotent” refers to a capacity to grow and develop into a normal plant. Totipotent plant tissue has both the complete genetic information of a plant and the ready capacity to develop into a complete plant if cultured under favorable conditions.

As used herein, the term “transcription factor” refers to a cytoplasmic or nuclear protein that binds to a gene, or binds to an RNA transcript of a gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the terms “transformed”, “transgenic”, and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic”, or “non-recombinant” host refers to a wild-type organism, e.g., a bacterium or plant, that does not contain the heterologous nucleic acid molecule.

### III. Nucleic Acid Sequences

The presently disclosed subject matter pertains in some embodiments to novel methods for the stable transformation of soybean cells with nucleic acid sequences of interest and to the regeneration of transgenic soybean plants.

Accordingly, the methods of the presently disclosed subject matter can be employed to express any nucleic acid of interest in soybean plants. A gene of interest can include, but is not limited to, a gene for herbicide resistance, disease resistance, or insect/pest resistance, or can be a selectable or scorable marker, and can comprise a plant-operable promoter, a coding region, and a 3′ terminator region. Further, the foreign nucleic acid can include DNA, RNA, and combinations thereof to be inserted into the plant to produce a transformant. In some embodiments, the foreign nucleic acid comprises one or more genes that are contained in a plasmid. Plasmids containing heterologous nucleic acid are available commercially, or can be created in vitro using conventional methods of recombinant DNA manipulation. The plasmid can then be introduced into the vector using conventional methods. The specific nucleic acid can be selected according to the desired properties of the transformant.

Herbicide resistance genes suitable for use in conjunction with the disclosed methods can include, but are not limited to, the AHAS gene for resistance to imidazolinone or sulfonyl urea herbicides, the pat or bar gene for resistance to bialaphos or glufosinate, the EPSP synthase gene for resistance to glyphosate, and so forth. Disease resistance genes can include, but are not limited to, genes for antibiotic synthetic enzymes, e.g., for pyrrolnitrin synthetic enzymes, plant derived resistance genes, and the like. Insect resistance genes can include, but are not limited to, genes for insecticidal proteins from Bacillus thuringiensis and the like. Genes of interest can also encode enzymes involved in biochemical pathways, the expression of which alters a trait that is important in food, feed, nutraceutical, and/or pharmaceutical production.

In accordance with the presently disclosed subject matter, the nucleic acid to be transferred can be contained within an expression cassette. The expression cassette can comprise a transcriptional initiation region linked to a nucleic acid or gene of interest. Such an expression cassette can be provided with a plurality of restriction sites for insertion of the gene or genes of interest (e.g., one gene of interest, two genes of interest, etc.) to be under the transcriptional regulation of the regulatory regions. In some embodiments of the presently disclosed subject matter, the nucleic acid to be transferred contains two or more expression cassettes, each of which encodes at least one gene of interest.

The transcriptional initiation region, (e.g., the promoter) can be native or heterologous to the host. Any suitable promoter known in the art can be employed according to the presently disclosed subject matter (including bacterial, yeast, fungal, insect, mammalian, and plant promoters). Exemplary promoters include, but are not limited to, the Cauliflower Mosaic Virus 35S promoter, the opine synthetase promoters (e.g., nos, mas, ocs, etc.), the ubiquitin promoter, the actin promoter, the ribulose bisphosphate (RubP) carboxylase small subunit promoter, and the alcohol dehydrogenase promoter. Other promoters from viruses that infect plants can also be suitable in the presently disclosed methods including, but not limited to, promoters isolated from Dasheen mosaic virus, Chlorella virus (e.g., the Chlorella virus adenine methyltransferase promoter), tomato spotted wilt virus, tobacco rattle virus, tobacco necrosis virus, tobacco ring spot virus, tomato ring spot virus, cucumber mosaic virus, peanut stump virus, alfalfa mosaic virus, and the like.

As would be understood by one of ordinary skill in the art upon a review of the present disclosure, promoters can be chosen to give a desired level of regulation. For example, in some instances, it can be advantageous to use a promoter that confers constitutive expression (e.g, the ubiquitin promoter, the RubP carboxylase gene family promoters, or the actin gene family promoters). Alternatively, in some embodiments, it can be advantageous to use promoters that are activated in response to specific environmental stimuli (e.g., heat shock gene promoters, drought-inducible gene promoters, pathogen-inducible gene promoters, wound-inducible gene promoters, and light/dark-inducible gene promoters) or plant growth regulators (e.g., promoters from genes induced by abscissic acid, auxins, cytokinins, and gibberellic acid). In some embodiments, promoters can be chosen that give tissue-specific expression (e.g., root, leaf and floral-specific promoters).

The transcriptional cassette can comprise in the 5′-3′ direction of transcription, a transcriptional and translational initiation region, a nucleotide sequence of interest, and a transcriptional and translational termination region functional in plants. Any suitable termination sequence known in the art can be used in accordance with the presently disclosed subject matter. The termination region can be native to the transcriptional initiation region, native to the nucleotide sequence of interest, or can be derived from another source. In some embodiments, termination regions can be used from the Ti-plasmid of Agrobacterium tumefaciens, such as the octopine synthetase and nopaline synthetase termination regions. See, Guerineau et al. (1991) Mol. Gen. Genet. 262: 141; Proudfoot (1991) Cell 64: 671; Sanfacon et al. (1991) Genes Dev. 5:141; Mogen et al. (1990) Plant Cell 2:1261; Munroe et al. (1990) Gene 91: 151; Ballas et al. (1989) Nucleic Acids Res. 17: 7891; and Joshi et al. (1987) Nucleic Acids Res. 15: 9627. Additional termination sequences that can be used in the presently disclosed subject matter are the pea RubP carboxylase small subunit termination sequence and the Cauliflower Mosaic Virus 35S termination sequence. Other suitable termination sequences will be apparent to those of ordinary skill in the art.

Alternatively, in some embodiments, the genes of interest can be provided on any other suitable expression cassette known in the art. Where appropriate, the genes can be optimized for increased expression in the transformed plant. Where mammalian, yeast, bacterial or plant dicot genes are used in the presently disclosed subject matter, they can be synthesized using monocot or soybean preferred codons for improved expression. Methods are available in the art for synthesizing plant preferred genes. See, e.g., U.S. Pat. No. 5,380,831; U.S. Pat. No. 5,436,391; and Murray et al. (1989) Nucleic Acids. Res. 17: 477; herein incorporated by reference in their entireties.

The expression cassettes can additionally contain 5′ leader sequences. Such leader sequences can act to enhance translation. Translation leaders are known in the art and can include, but are not limited to, picornavirus leaders (e.g., EMCV leader), potyvirus leaders, human immunoglobulin heavy-chain binding protein untranslated leader from the coat protein mRNA of alfalfa mosaic virus, tobacco mosaic virus leader, and maize chlorotic mottle virus leader. Other methods known to enhance translation can also be utilized, e.g., introns and the like.

The expression cassettes can contain more than one gene or nucleic acid sequence to be transferred and expressed in the transformed plant. Thus, each nucleic acid sequence can be operably linked to 5′ and 3′ regulatory sequences. Alternatively, multiple expression cassettes can be provided.

In some embodiments, the expression cassette can comprise a selectable marker gene for the selection of transformed cells. Selectable marker genes can be utilized for the selection of transformed cells or tissues. Selectable marker genes can include, but are not limited to, genes encoding antibiotic resistance, such as those encoding neomycin phosphotransferase II (NEO) and hygromycin phosphotransferase (HPT), as well as genes conferring resistance to herbicidal compounds. Herbicide resistance genes can code for a modified target protein insensitive to the herbicide or for an enzyme that degrades or detoxifies the herbicide in the plant before it can act. See, for example, DeBlock et al. (1987) EMBO J. 6: 2513); DeBlock et al. (1989) Plant Physiol. 91: 691; Fromm et al. (1990) BioTechnology 8: 833; Gordon-Kamm et al. (1990) Plant Cell 2: 603. For example, resistance to glyphosphate or sulfonylurea herbicides can been obtained using genes coding for the mutant target enzymes 5-enolpyruvylshikimate-3-phosphate synthase and acetolactate synthase. Further, resistance to glufosinate ammonium, boromoxynil, and 2,4-dichlorophenoxyacetate (2,4-D) can be accomplished by using bacterial genes encoding phosphinothricin acetyltransferase, a nitrilase, or a 2,4-dichlorophenoxyacetate monooxygenase, which detoxify the respective herbicides.

For purposes of the presently disclosed subject matter, selectable marker genes include, but are not limited to, genes encoding: hygromycin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, mannose, glyphosate, sulfonylurea, imidazolinone, glufosinate, phenoxy proprionic acid, cycloshexone, triazine, benzonitrile, and combinations thereof.

The PMI gene confers resistance to mannose and could be used as a selectable marker. As noted above, other selectable markers that could be used in the vector constructs include, but are not limited to the ALS gene for imidazolinone resistance, the HPH or HYG gene for hygromycin resistance, the EPSP synthase gene for glyphosate resistance, the Hml gene for resistance to the Hc-toxin, and other selective agents used routinely and known to one of ordinary skill in the art. See, for example, Yarranton (1992) Curr. Opin. Biotech 3: 506 (1992); Yao et al. (1992) Cell 71: 63; Reznikoff (1992) Mol. Microbiol. 6: 2419; Hu et al. (1987) Cell 48, 555; Brown et al. (1987) Cell 49: 603; Figge et al. (1988) Cell 52: 713; Deuschle et al. (1989) Proc. Natl. Acad. Sci. USA 86: 5400; Fuerst et al. (1989) Proc. Natl. Acad. Sci. USA 86: 2549; Deuschle et al. (1990) Science 248: 480, herein incorporated by reference. The above list of selectable marker genes are not meant to be limiting, and any selectable marker gene can be used in the presently disclosed subject matter.

Where appropriate, the selectable marker genes and other genes and nucleic acids of interest to be transferred can be synthesized for optimal expression in soybean cells. Particularly, the coding sequence of the genes can be modified to enhance expression in soybean cells. The synthetic nucleic acid can be designed to be expressed in the transformed tissues and plants at a higher level. Accordingly, the use of optimized selectable marker genes can result in higher transformation efficiency.

Additional sequence modifications are known in the art to enhance gene expression in a cellular host. Particularly, sequences encoding spurious polyadenylation signals, exon-intron splice site signals, transposon-like repeats, and other such well-characterized sequences that can be deleterious to gene expression can be eliminated. In some embodiments, the G-C content of the sequence can be adjusted to levels average for a given cellular host, as calculated by reference to known genes expressed in the host cell. When desired, the sequence can be modified to avoid predicted hairpin secondary mRNA structures.

### IV. Target Tissues

A starting material for the transformation methods disclosed herein is an immature soybean cotyledon, which can be isolated from a growing soybean plant. Various soybean varieties are suitable for use in the presently disclosed subject matter. For example, the soybean Glycine max (L.) Merril varieties include, but are not limited to, Jack, S42-H1, and 03JR101915. In some embodiments, the immature soybean cotyledons are isolated from greenhouse-grown soybean plants. As would be apparent to one of ordinary skill in the art, the soybean plants can be grown using standard conditions for the successful growth of plants, for example, grown under a photoperiod of about 14/10 hours of daylight/dark at about at about 28° C.

Soybean seed pods can be isolated from the plants using any desired technique, including but not limited to, removal of the seed pod using a gloved hand, removal using any of a number of mechanical devices, and the like. The soybean embryo can be removed from the seed pod using any desired technique, including but not limited to, excising the seed from the seed pod and gently squeezing the embryo out of the seed coat using forceps and a scalpel. The seed pods can be sterilized after removal from the plant using standard techniques, including but not limited to, rinsing with water, diluted chlorine bleach, and/or alcohol one or more times.

As would be readily understood by one of skill in the art, seed storage can be accomplished by any of a variety of known methods. For example, the seeds can be stored at 4° C. for later use.

After extraction from the pods, the immature soybean cotyledons can be pre-cultured on liquid or solid medium. In some embodiments, the medium comprises MS salts and B5 vitamins. In some embodiments, the medium comprises an auxin, including but not limited to, 2,4-D. In some embodiments, the medium can be D40 medium or D20 medium. The cotyledons can be pre-cultured for at least about 8 hours, in some embodiments at least about 12 hours. In some embodiments, the immature cotyledons can be pre-cultured for up to about 5 days.

V. Agrobacterium-Mediated Transformation

Agrobacterium-mediated gene transfer exploits the natural ability of Agrobacterium to transfer DNA into plant chromosomes. As is well-known in the art, Agrobacterium is a plant pathogen that can transfer a set of genes into plant cells. In some embodiments of the presently disclosed subject matter, immature soybean cells can be transformed using Agrobacterium tumefaciens.

Those skilled in the art will appreciate that the disclosed methods apply equally well to Agrobacterium rhizogenes. Transformation using Agrobacterium rhizogenes has developed analogously to that of Agrobacterium tumefaciens and has been successfully utilized to transform plants, including but not limited to, alfalfa, Solanum nigrum L., and poplar. See, for example, Hooykaas, Plant Mol. Biol. (1989) 13: 327; Smith et al., Crop Science (1995) 35: 301 (1995); Chilton, Proc. Natl. Acad. Sci. USA (1993) 90: 3119; Mollony et al., Monograph Theor. Appl. Genet NY (1993) 19: 148; Ishida et al. Nature Biotechnol. (1996) 14: 745 (1996); and Komari et al., The Plant Journal (1996) 10:165 (1996), the disclosures of which are incorporated herein by reference.

Soybean explants can be prepared using any of a variety of methods. Particularly, in some embodiments, preparing the explant can comprise wounding the explant. As would be readily understood by one of skill in the art upon review of the instant disclosure, wounding can comprise any injury to the tissue of the explant. In some embodiments, the wounding can comprise one or more cuts, stabs, lacerations, lesions or traumas inflicted to the tissue of an explant. In some embodiments, the wounding can be inflicted by a mechanical instrument (such as, but not limited to, a scalpel blade).

Thus, in some embodiments, wounding of the explant tissue can be used to facilitate gene transfer. Accordingly, in some embodiments, a wound can be created on the abaxial and/or adaxial side of the explant tissue. Many methods of wounding can be used, including but not limited to, cutting, abrading, piercing, poking, penetrating with fine particles or pressurized fluids, plasma wounding, applying of hyperbaric pressure, and/or sonicating. Wounding can be performed using objects such as, but not limited to, scalpels, scissors, needles, abrasive objects, airbrush, particles, vacuum infiltration, electric gene guns, or sound waves.

In addition, in some embodiments, wounding of the explant tissue can be performed by bombarding the explant tissue with a microprojectile. In some embodiments, the wound can be created on the abaxial and/or adaxial side of the explant tissue. According to some embodiments of the presently disclosed subject matter, the bombardment comprises the steps of providing a soybean immature cotyledon tissue as a target, and propelling a microprojectile at the soybean tissue at a velocity sufficient to wound the cells within the tissue. As would be apparent to one of skill in the art upon review of the instant disclosure, any ballistic cell transformation apparatus can be used in practicing the presently disclosed subject matter. See, for example, Sanford et al. (Particulate Science and Technology (1988) 5:27), Klein et al. (Nature (1987) 327:70), and in European Patent Application No. EP 270,356.

The microprojectile can be formed from any material having sufficient density and cohesiveness to be propelled through the cell wall, given the particle's velocity and the distance the particle must travel. Examples of materials suitable for making microprojectiles include, but are not limited to, metal (e.g., gold), glass, silica, ice, polyethylene, polypropylene, polycarbonate, and carbon compounds (e.g., graphite, diamond). The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical.

The Agrobacterium-mediated transformation process of the presently disclosed subject matter can comprise several steps. A representative embodiment can include, but is not limited to, an inoculation step and a co-cultivation step. In some embodiments, these steps are followed by a selection step, and in some embodiments by a selection and a regeneration step, as discussed in detail hereinbelow.

In the inoculation step, the soybean cells to be transformed are exposed to Agrobacterium. In some embodiments, the cells are brought into contact with the Agrobacterium in a liquid medium. In some embodiments, the cells are brought into contact with the Agrobacterium in a solid medium. In some embodiments, the Agrobacterium can be modified to contain a gene or nucleic acid of interest, wherein the nucleic acid can be inserted into the vector.

Agrobacterium containing a plasmid of interest can be maintained on Agrobacterium master plates and stock frozen at about −80° C. Master plates can then be used to inoculate agar plates to obtain Agrobacterium that is then resuspended in medium for use in the infection process. Alternatively, bacteria from the master plate can be used to inoculate broth cultures that are grown to logarithmic phase prior to transformation.

Concentrations of Agrobacterium employed in the methods of the presently disclosed subject matter can vary depending on the Agrobacterium strain utilized, the tissue being transformed, the soybean species being transformed, and the like. To optimize the transformation protocol for a particular soybean species or tissue, the tissue to be transformed can be incubated with various concentrations of Agrobacterium. Likewise, the level of marker gene expression and the transformation efficiency can be assessed for various Agrobacterium concentrations. While the concentration of Agrobacterium can vary, generally a concentration range of about 1×10³ cfu/ml to about 1×10¹⁰ cfu/ml can be employed in the methods of the presently disclosed subject matter. In some embodiments, the concentration of Agrobacterium can vary from about 1×10³ cfu/ml to about 1×10⁹ cfu/ml. In some embodiments, the concentration of Agrobacterium can vary from about 1×10⁸ to about 1×10⁹ cfu/ml.

The soybean tissue to be transformed can generally be added to the Agrobacterium suspension in a liquid or solid contact phase containing a concentration of Agrobacterium to optimize transformation efficiencies. The contact phase facilitates maximum contact of the tissue to be transformed with the suspension of Agrobacterium. Inoculation generally can be allowed to proceed for about 30 to 45 minutes.

In some embodiments, one or more virulence-enhancing compounds (such as, but not limited to, acetosyringone) can be added to enhance gene delivery. Furthermore, to enhance transformation frequency, in some embodiments, tissue can be cultured in medium containing antioxidants including, but not limited to, cysteine. As further alternatives, tissue wounding, vacuum pressure, or cultivation in medium containing acetosyringone or other suitable agent can be employed to promote the transformation efficiency.

In some embodiments, after inoculation, the bacteria can be removed from the explants and replaced with culture medium containing a herbicide, including but not limited to, D40 culture medium. Explants can then be resuspended and plated onto suitable plates such as Gelman plates.

For Agrobacterium-mediated transformation, the explants can be co-cultured for a time with the Agrobacterium in order to increase transformation efficiency. In the co-cultivation step, the majority of the Agrobacterium cells are removed, such as by pouring or pipetting, and the explants are co-cultivated with the remainder of the Agrobacterium. Particularly, in the co-cultivation step, the soybean explants can be co-cultivated with Agrobacterium on a co-cultivation medium. In some embodiments, the soybean explants can be co-cultivated with the Agrobacterium for about 2 to 5 days. In some embodiments, co-cultivation can be carried out in the dark at 23° C. to enhance the transformation efficiency. Additionally, as described herein above for the inoculation step, co-culturing can be done on medium containing acetosyringone or other suitable agent to promote transformation efficiency. In some embodiments, the co-culturing step can be performed in the presence of cytokinins, which can act to enhance cell proliferation.

In some embodiments, after the co-cultivation step, excess bacteria are removed from the explants by washing in culture medium, including but not limited to, D40 culture medium. In some embodiments, the culture medium comprises antibiotics, including but not limited to, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, vancomycin, and combinations thereof. In some embodiments, the excess bacteria are removed by blotting with filter paper, washing, decanting excess bacteria, and the like.

Following the co-cultivation step, or resting/decontamination step, transformants can be selected and soybean plants regenerated as described herein below.

### VI. Transformation of Immature Cotyledons by Ballistic Bombardment

In some embodiments, the presently disclosed subject matter comprises a method of transforming immature soybean cotyledons with a nucleotide sequence of interest using a microprojectile.

According to some embodiments of the presently disclosed subject matter, the ballistic transformation method comprises the steps of providing a soybean immature cotyledon tissue as a target, prepared as described herein above, and propelling the microprojectile carrying the nucleotide sequence at the soybean tissue at a velocity sufficient to pierce the walls of the cells within the tissue and to deposit the nucleotide sequence within a cell of the tissue to thereby provide a transformed tissue. In some embodiments of the presently disclosed subject matter, the method further includes culturing the transformed tissue with a selection agent, as described herein below. In some embodiments, the selection step is followed by the step of regenerating transformed soybean plants from the transformed tissue.

As would be apparent to one of skill in the art, any ballistic cell transformation apparatus can be used in practicing the presently disclosed subject matter. In some embodiments, a commercially-available helium gene gun (PDS-1000/He) manufactured by DuPont (Wilmington, Del., United States of America) can be employed. Alternately, an apparatus configured as described by Klein et al. (Nature (1987) 327:70) can be utilized, comprising, in some embodiments, a bombardment chamber, which is divided into two separate compartments by an adjustable-height stopping plate.

The microprojectile can be formed from any material having sufficient density and cohesiveness to be propelled through the cell wall, given the particle's velocity and the distance the particle must travel. Examples of materials suitable for making microprojectiles include, but are not limited to, metal, glass, silica, ice, polyethylene, polypropylene, polycarbonate, and carbon compounds (e.g., graphite, diamond). The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles can enhance their DNA carrying capacity.

In some embodiments, the nucleotide sequence can be immobilized on the particle by precipitation. The precise precipitation parameters employed can vary depending upon factors such as the particle acceleration procedure employed, as is well known in the art. The carrier particles can optionally be coated with an encapsulating agent such as polylysine to improve the stability of nucleotide sequences immobilized thereon.

In some embodiments, ballistic transformation is achieved without use of microprojectiles. For example, an aqueous solution containing the nucleotide sequence of interest as a precipitate can be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described hereinabove). Other approaches include placing the nucleic acid precipitate itself (“wet” precipitate) or a freeze-dried nucleotide precipitate directly on the plate-contact end of the macroprojectile without a microprojectile. In some embodiments, the nucleotide sequence can be propelled at the tissue target in the absence of a microprojectile.

After ballistic bombardment of the target tissue, transformants can be selected and soybean plants regenerated as described hereinbelow.

### VII. Post-Transformation Explant Growth

As discussed in detail hereinabove, soybean tissue can be transformed according to the presently disclosed subject matter (including but not limited to ballistic bombardment or Agrobacterium-mediated transformation). After the transformation step, the transformed tissue can be exposed to selective pressure to select for those cells that have received and are expressing the polypeptide from the heterologous nucleic acid introduced by the expression cassette. The agent used to select for transformants can select for preferential growth of cells containing at least one selectable marker insert positioned within the expression cassette and delivered by ballistic bombardment or by Agrobacterium.

In some embodiments, the transformed tissue can be subjected to an optional resting and decontamination step. For the resting/decontamination step, the transformed cells can be transferred to a medium in the absence of any selective pressures to permit recovery and proliferation of transformed cells containing the heterologous nucleic acid.

The resting/decontamination step can be carried out for as long as is necessary to inhibit the growth of Agrobacterium and to increase the number of transformed cells prior to selection. In some embodiments, the resting/decontamination step can be carried out for up to about 2 weeks. In some embodiments, the resting phase is performed in the absence of any selective pressures to permit recovery and proliferation of transformed cells containing the heterologous nucleic acid. In some embodiments, an antibiotic is added to the recovery medium to kill or inhibit Agrobacterium growth.

In some embodiments, after explants are transformed by Agrobacterium-mediated methods, the explants can be transferred to recovery medium, such as but not limited to, D40 medium, to induce growth. In some embodiments, the recovery medium can contain hormones inducing embryogenesis (such as but not limited to 2,4-D).

In some embodiments, an antibiotic is added to the recovery medium to kill or inhibit Agrobacterium growth. Representative antibiotics are known in the art, including but not limited to, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and the like. Concentrations of the antibiotic can vary according to what is standard for each antibiotic. For example, concentrations of carbenicillin can range from about 50 mg/l to about 250 mg/l carbenicillin in solid media. Those of ordinary skill in the art will recognize that the concentration of antibiotic can be optimized for a particular transformation protocol without undue experimentation.

In some embodiments, explants can remain in recovery medium for as long as is necessary to inhibit the growth of Agrobacterium and to increase the number of transformed cells prior to selection. In some embodiments, the explants can remain in the recovery medium for 5 to 10 days.

In some embodiments, the explants are then transferred to selection medium, including but not limited to D40 medium, supplemented with antibiotics, including but not limited to, hygromycin for about 30 days.

In some embodiments, the proliferating soybean callus can then be transferred to selection medium, including but not limited to, D20 medium supplemented with antibiotics for about 2 months. Representative antibiotics are known in the art, including but not limited to, hygromycin, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and the like. Concentrations of the antibiotic can vary according to what is standard for each antibiotic.

As would be appreciated by one of skill in the art upon a review of the present disclosure, selection can be carried out long enough to kill non-transformants and to allow transformed cells to proliferate at a similar rate to non-transformed cells. Thus, in some embodiments, the selection period can be longer with cells that proliferate at a slower rate.

The resistant clones can then be transferred to embryo differentiation medium, including but not limited to MSM6 medium, supplemented with activated charcoal and antibiotics, for about 3 weeks. Representative antibiotics are known in the art, including but not limited to, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and the like. Concentrations of the antibiotic can vary according to what is standard for each antibiotic.

In some embodiments, the resistant clones can then be transferred for about 2 weeks to culture medium, including but not limited to, MSM6 medium supplemented with antibiotics. Representative antibiotics are known in the art, including but not limited to, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and the like. Concentrations of the antibiotic can vary according to what is standard for each antibiotic.

Matured embryos can then be dessicated for about 3 to 5 days and germinated on germination medium, including but not limited to, MS-0 medium.

Well developed seedlings with leaves and roots can then be transferred to rooting medium supplemented with cell growth regulating compounds, including but not limited to, auxins (such as but not limited to IAA, NAA, and IBA), cytokinins (such as but not limited to thidiazuron, kinetin, BAP, Zeatin, and isopentenyl adenine) and/or gibberellic acids (GA₃).

During the rooting process, any method known in the art can be utilized to verify that the rooted plants are transformed with the transferred nucleic acid of interest. For example, histochemical staining, ELISA assay, Southern hybridization, Northern hybridization, Western immunoblotting, PCR, Taqman assay, and the like can be used to detect the transferred nucleic acids or protein in the rooted plants. In some embodiments, leaves can be sampled for analysis to identify transformants. Particularly, a portion of the plant sample can be assayed for the presence of the foreign nucleic acid or the protein that such nucleic acid encodes. Positives are rooted and transplanted to soil and grown in greenhouse to fully mature and for seeds.

### VIII. Transgenic Plants and Seeds

Transgenic plants comprising a heterologous nucleic acid (i.e., comprising cells or tissues transformed in accordance with the methods described herein), as well as the seeds and progeny produced by the transgenic plants, are an additional aspect of the presently disclosed subject matter. Procedures for cultivating transformed cells to useful cultivars are known to those skilled in the art. Techniques are known for the in vitro culture of plant tissue, and in a number of cases, for regeneration into whole plants. In some embodiments, the presently disclosed subject matter comprises transgenic plant tissue, plants, or seeds containing the nucleic acids described above.

As provided hereinabove, seeds and progeny plants of the regenerated plants can comprise an aspect of the presently disclosed subject matter. Accordingly, the term “seeds” can encompass seeds of the transformed plant, as well as seeds produced from the progeny of the transformed plants. Plants of the presently disclosed subject matter can include not only the transformed and regenerated plants, but also progeny of transformed and regenerated plants produced by the methods described herein.

Plants produced by the described methods can be screened for successful transformation by standard methods described above. Seeds and progeny plants of regenerated plants of the presently disclosed subject matter can be continuously screened and selected for the continued presence of the transgenic and integrated nucleic acid sequence in order to develop improved plant and seed lines, which are another aspect of the presently disclosed subject matter. Desirable transgenic nucleic acid sequences can thus be moved (i.e., introgressed or inbred) into other genetic lines such as certain elite or commercially valuable lines or varieties. Methods of introgressing desirable nucleic acid sequences into genetic plant lines can be carried out by a variety of techniques known in the art, including by classical breeding, protoplast fusion, nuclear transfer and chromosome transfer. Breeding approaches and techniques are known in the art, and are set forth in, for example, J. R. Welsh, Fundamentals of Plant Genetics and Breeding (John Wiley and Sons, New York, (1981)); Crop Breeding (D. R. Wood, ed., American Society of Agronomy, Madison, Wis., (1983)); O. Mayo, The Theory of Plant Breeding, Second Edition (Clarendon Press, Oxford, England (1987)); and Wricke and Weber, Quantitative Genetics and Selection Plant Breeding (Walter de Gruyter and Co., Berlin (1986)). Using these and other techniques in the art, transgenic plants and inbred lines obtained according to the presently disclosed subject matter can be used to produce commercially valuable hybrid plants and crops, which hybrids are also an aspect of the presently disclosed subject matter.

### EXAMPLES

The following Examples have been included to illustrate representative and exemplary modes of the presently disclosed subject matter. In light of the present disclosure and the general level of skill in the art, those of skill will appreciate that the following Examples are intended to be exemplary only and that numerous changes, modifications, and alterations can be employed without departing from the spirit and scope of the presently disclosed subject matter.

### Materials and Methods

Media used in the Agrobacterium-mediated transformation protocol employed to develop transformed soybean plants were prepared using standard methods known to one of ordinary skill in the art. The following media were used in the Examples described herein.

### D40 Liquid Medium

4.3 g of MS basal salt mixture, B vitamins [100 mg of myo-Inositol, 1 mg of nicotinic acid, 1 mg of pyridoxine HCl and 10 mg of thiamine HCl], 30 g sucrose, and 40 mg 2,4-D were combined and taken up to a final volume of 1 L using sterile water. The pH was adjusted to 7.0.

### D40 Solid Medium 1

4.3 g of MS basal salt mixture, B vitamins [100 mg of myo-Inositol, 1 mg of nicotinic acid, 1 mg of pyridoxine HCl and 10 mg of thiamine HCl], 30 g sucrose, 40 mg 2,4-D, and 2 g Gelrite (Merck & Co., Inc., Rahway, N.J., United States of America) were combined and taken up to a final volume of 1 L using sterile water. The pH was adjusted to 7.0.

### D40 Solid Medium 2

4.3 g of MS basal salt mixture, B vitamins [100 mg of myo-Inositol, 1 mg of nicotinic acid, 1 mg of pyridoxine HCl and 10 mg of thiamine HCl], 10 mg hygromycin, 30 g sucrose, 40 mg 2,4-D, and 2 g Gelrite (Merck & Co., Inc., Rahway, N.J., United States of America) were combined and taken up to a final volume of 1 L using sterile water. The pH was adjusted to 7.0.

### D40 Solid Medium 3

4.3 g of MS basal salt mixture, B vitamins [100 mg of myo-Inositol, 1 mg of nicotinic acid, 1 mg of pyridoxine HCl and 10 mg of thiamine HCl], 10 g mannose, 20 g sucrose, 40 mg 2,4-D, and 2 g Gelrite (Merck & Co., Inc., Rahway, N.J., United States of America) were combined and taken up to a final volume of 1 L using sterile water. The pH was adjusted to 7.0.

### D20 Medium 1

4.3 g of MS basal salt mixture, B vitamins [100 mg of myo-Inositol, 1 mg of nicotinic acid, 1 mg of pyridoxine HCl and 10 mg of thiamine HCl], 10 mg hygromycin, 30 g sucrose, and 20 mg 2,4-D were combined and taken up to a final volume of 1 L] using sterile water. The pH was adjusted to 5.6.

### D20 Medium 2

4.3 g of MS basal salt mixture, B vitamins [100 mg of myo-Inositol, 1 mg of nicotinic acid, 1 mg of pyridoxine HCl and 10 mg of thiamine HCl], 25 mg hygromycin, 30 g sucrose, and 20 mg 2,4-D were combined and taken up to a final volume of 1 L] using sterile water. The pH was adjusted to 5.6.

### D20 Medium 3

4.3 g of MS basal salt mixture, B vitamins [100 mg of myo-Inositol, 1 mg of nicotinic acid, 1 mg of pyridoxine HCl and 10 mg of thiamine HCl], 15 g mannose, 20 g sucrose, and 20 mg 2,4-D were combined and taken up to a final volume of 1 L using sterile water. The pH was adjusted to 5.6.

### D20 Medium 4

4.3 g of MS basal salt mixture, B vitamins [100 mg of myo-Inositol, 1 mg of nicotinic acid, 1 mg of pyridoxine HCl and 10 mg of thiamine HCl], 20 g mannose, 15 g sucrose, and 20 mg 2,4-D were combined and taken up to a final volume of 1 L using sterile water. The pH was adjusted to 5.6.

### SoyAIM-EV Inoculation Medium

20 g sucrose, 4 g MES, and 40 mg acetosyringone were combined and taken up to a final volume of 1 L using sterile water. The pH was adjusted to 5.4.

### MSM6 Medium

4.3 g MS salts, B5 vitamins [100 mg of myo-Inositol, 1 mg of nicotinic acid, 1 mg of pyridoxine HCl and 10 mg of thiamine HCl], 60 g maltose and 2 g Gelrite were taken up to a final volume of 1 L using sterile water. The pH was adjusted to 5.8.

### MSM6+AC Medium

4.3 g MS salts, B5 vitamins [100 mg of myo-Inositol, 1 mg of nicotinic acid, 1 mg of pyridoxine HCl and 10 mg of thiamine HCl], 60 g maltose, 5 g activated charcoal and 2 g Gelrite were taken up to a final volume of 1 L using sterile water. The pH was adjusted to 5.8.

### MS-O Medium

4.3 g MS salts, B5 vitamins [100 mg of myo-Inositol, 1 mg of nicotinic acid, 1 mg of pyridoxine HCl and 10 mg of thiamine HCl], 30 g sucrose, and 2 g Gelrite were taken up to a final volume of 1 L using sterile water. The pH was adjusted to 5.8.

### Rooting Medium

4.3 g of MS salt, B5 vitamins [100 mg of myo-Inositol, 1 mg of nicotinic acid, 1 mg of pyridoxine HCl and 10 mg of thiamine HCl], 100 mg of glutamine, 100 mg of asparagine, 5 g sucrose, 0.7 mg of IBA and 2 g Gelrite were taken up to a final volume of 1 L using sterile water. The pH was adjusted to 5.6.

### Example 1

### Isolation of Immature Cotyledons from Seed Pods

Soybean (Glycine max cultivars Jack, 03JR101915, or S42H1) stock plants were grown in a greenhouse under 14 hours of daylight at 28° C. Immature pods were collected 2-3 weeks after flower formation and sterilized by immersing in 30% chlorine bleach (available under the registered trademark CHLOROX®) for 20 minutes. Sterilized pods were then rinsed thoroughly with sterile water.

Pods were opened by cutting the edge of the pod with a sterile scalpel. Immature zygotic embryos were isolated from the seeds by cutting off the seed coat at the basis of the seed and gently squeezing the embryo out. The embryo axis was discarded and the immature cotyledons 2 to 8 mm in size were plated onto D40 or D20 medium for pre-culture overnight.

### Example 2

### Transformation Vector and Agrobacterium Strains

Agrobacterium tumefaciens transformation vectors were constructed using standard molecular techniques known in the art. The plasmid construct pBSC11369 was used, containing the ZsGreen and Hyg genes under control of a CMP promoter. The plasmids were introduced into Agrobacterium strains EHA101, EHA101+pAD1289, LBA4404, and LBA4404+pSB1.

Overnight cultures of the Agrobacterium strain containing the plasmid were grown for two days on plates with YP medium with appropriate antibiotics [100 mg/L spectinomycin and 50 mg/L kanamycin for EHA101; 100 mg/L spectinomycin, 50 mg/L kanamycin, 100 mg/L carbenicillin for EHA101+pAD1289; 100 mg/L spectinomycin for LBA4404 and 100 mg/L spectinomycin and 10 mg/L tetracycline for LBA4404+pSB1].

### Example 3

### Preparation of Agrobacterium for Transformation

Agrobacterium culture was initiated weekly from glycerol stock at −80° C. onto YP semi-solid medium containing appropriate antibiotics and grown at 28° C. in an incubator.

The Agrobacterium was streaked onto fresh YP medium containing appropriate antibiotics the day before the inoculation and was grown in a 28° C. incubator. For plant transformation use, the Agrobacterium was collected from the plate using a disposable plastic inoculation loop and suspended in liquid inoculation medium, such as SoyAIM-EV, in a sterile 50 ml disposable polypropylene centrifugation tube. The tube was shaken gently on a rotary shaker for about 30 to 60 minutes until the Agrobacterium cells were uniformly dispersed in the suspension. The Agrobacterium suspension was then diluted to an OD₆₆₀ of 0.1 to 0.2, and vortexed for about 15 seconds.

### Example 4

### Pre-Culture of Soybean Cotyledon Explants

Prior to wounding and infection with Agrobacterium, the soybean cotyledon explants of Example 1 were pre-cultured, whereby the explants were plated onto D40 solid medium 1 for at least about 8 to 12 hours, up to about 5 days.

### Example 5

### Wounding of Soybean Cotyledon Explants

Prior to inoculation with Agrobacterium, the immature cotyledons were wounded by different methods, as set forth in Examples 5.1, 5.2, 5.3 and 5.4.

### Example 5.1

Abaxial or adaxial sides of larger cotyledons (5-8 mm) and smaller cotyledons (2-5 mm) were wounded with a scalpel and/or needle.

### Example 5.2

Abaxial or adaxial sides of both larger cotyledons (5-8 mm) and smaller cotyledons (2-5 mm) were bombarded with gold particles (biolistic wounding).

### Example 5.3

Larger cotyledons (5-8 mm) and smaller (2-5 mm) were submerged to 5% whiskers solution and vortexed for 5 minutes.

### Example 5.4

Abaxial or adaxial sides of larger cotyledons (5-8 mm) and smaller cotyledons (2-5 mm) were wounded with a plurality of Acupuncture needles.

### Example 6

### Infection and Co-Cultivation of Soybean Cotyledon Explants

The wounded explants from Examples 5.1, 5.2, 5.3 and 5.4 were infected with Agrobacterium by mixing the explants with bacterial suspension as prepared in Example 3. The mixture was incubated with the prepared explants for about 30 to 45 minutes at room temperature.

Following infection, the Agrobacterium suspension was removed from the explants, and replaced with 0.5 to 1 mL of D40 liquid medium. The explants were resuspended and plated onto Gelman plates for co-cultivation. The plates were incubated for 2 to 5 days at 23° C. in the dark and then washed with liquid D40 medium for about 30 minutes.

The consistency of recovery of GFP-expressing embryos after wounding with gold particle bombardment is given in Table 1. From 20 experiments, the number of recovered transformation events ranged from 0 to 16, with an average number of recovered events per experiment of 5.7.

TABLE 1
The consistency of recovery of GFP-expressing embryos after
wounding with gold particle bombardment

| | Events |
|---|---|
| Experiment | recovered |
| 1 | 3 |
| 2 | 3 |
| 3 | 16 |
| 4 | 4 |
| 5 | 4 |
| 6 | 1 |
| 7 | 1 |
| 8 | 8 |
| 9 | 7 |
| 10 | 15 |
| 11 | 9 |
| 12 | 0 |
| 13 | 13 |
| 14 | 5 |
| 15 | 4 |
| 16 | 2 |
| 17 | 0 |
| 18 | 7 |
| 19 | 7 |
| 20 | 5 |
| Average | 5.7 |

The distribution of transgene copy numbers is given in Table 2. As shown in Table 2, approximately half of the explants had low copy numbers.

TABLE 2
The distribution of transgene copy numbers

| Copy numbers | Distribution |
|---|---|
| High | 19 |
| Medium | 29 |
| Low | 52 |

The transformation frequency (TF) of GFP-expressing embryos after pre-culture on D40 medium is given in Table 3.

TABLE 3
Formation of Transgenic GFP-Expressing Embryos
After Pre-culture on D40 Medium

| Experi- | | | | Explants | |
|---|---|---|---|---|---|
| ment | Agrobacterium | Con- | Explants | w/GFP | TF |
| ID | strain | struct | Plated | Embryos | (%) |
| IC-1.1 | EHA101 | 11369 | 20 | 3 | 15 |
| IC-1.2 | EHA101 + pAD1289 | 11369 | 20 | 1 | 5 |
| IC-1.3 | LBA4404 | 11369 | 20 | 4 | 20 |
| IC-1.4 | LBA4404 + pSB1 | 11369 | 20 | 4 | 20 |
| MS-22 | EHA101 | 11369 | 138 | 2 | 1.5 |
| MS-23 | LBA4404 + pSB1 | 11369 | 98 | 4 | 4.0 |
| MS-27 | LBA4404 + pSB1 | 11369 | 41 | 2 | 4.9 |
| MS-28 | LBA4404 + pSB1 | 11369 | 103 | 5 | 4.9 |
| MS-30 | EHA101 | 11369 | 43 | 6 | 1.4 |
| MS-37 | LBA4404 + pSB1 | 11369 | 72 | 2 | 2.3 |
| MS-38 | LBA4404 + pSB1 | 11369 | 212 | 2 | 0.9 |
| MS-40 | LBA4404 + pSB1 | 11369 | 159 | 1 | 1.3 |
| MS-43 | LBA4404 + pSB1 | 11369 | 108 | 1 | 0.9 |
| MS-45 | LBA4404 + pSB1 | 11369 | 156 | 2 | 1.3 |
| MS-47 | LBA4404 + pSB1 | 11369 | 87 | 2 | 2.3 |
| MS-52 | LBA4404 + pSB1 | 11369 | 32 | 1 | 3.1 |
| MS-55 | LBA4404 + pSB1 | 11369 | 151 | 2 | 1.3 |
| MS-56 | LBA4404 + pSB1 | 11369 | 177 | 6 | 3.1 |

### Example 7

### Regeneration and Selection of Transgenic Plants

After co-cultivation, the explants were transferred onto recovery medium with antibiotics to kill Agrobacterium or to inhibit Agrobacterium growth, without selection agent, such as D40 supplemented with 300 mg/L timentin and 250 mg/L of cefotaxime. The plates were incubated for 5 to 10 days.

### Example 7.1

The explants were then transferred to D40 solid medium 2 (10 mg/L hygromycin) supplemented with antibiotics for about 14 days.

The explants were then transferred to D20 medium 1 (10 mg/L hygromycin) for about 14 days.

The proliferating callus was then selected on D20 medium 2 (25 mg/L hygromycin) for about 2 months.

Resistant clones were transferred to embryo differentiation medium MSM6+Ac for about 3 weeks.

The clones were then transferred for about 2 weeks to MSM6 medium.

Matured embryos were then dessicated for about 3 to 5 days and germinated on MS-0 medium.

Well developed seedlings with leaves and roots were transferred to rooting medium supplemented with 0.7 mg/L IBA.

Leaves were sampled for TAQMAN analysis to identify transformants that contain the ZsGreen fluorescent protein gene. TAQMAN positive and rooted plants were rinsed with water to wash off the agar medium, and transplanted to soil and grown in the greenhouse for seeds.

### Example 7.2

The explants were then transferred to D40 solid medium 3 (10 g/L mannose and 20 g/L sucrose) supplemented with antibiotics for about 4 weeks.

The explants were then transferred to D20 medium 3 (15 g/L mannose and 20 g/L sucrose) for about 3 weeks.

The proliferating callus was then selected on D20 medium 4 (20 g/L mannose and 15 g/L sucrose) for about 2 months. Every three weeks the callus was sub-cultured to fresh selection medium.

Resistant clones were transferred to embryo differentiation medium MSM6+Ac for about 3 weeks.

The clones were then transferred for about 2 weeks to MSM6 medium.

Matured embryos were then desiccated for about 3 to 5 days and germinated on MS-0 medium.

Well developed seedlings with leaves and roots were transferred to rooting medium supplemented with 0.7 mg/L IBA.

Leaves were sampled for TAQMAN analysis to identify transformants that contain the ZsGreen fluorescent protein gene. TAQMAN positive and rooted plants were rinsed with water to wash off the agar medium, and transplanted to soil and grown in the greenhouse for seeds.

Table 4 is directed to the recovery of transgenic plants/event in Agrobacterium/Embryogenesis Transformation.

TABLE 4
Recovery of Transgenic Plants/Events in Agrobacterium/Embryogenesis
Transformation System

| | | | | | Transgenic | |
|---|---|---|---|---|---|---|
| | | | | Transgenic | Events | |
| Experiment | Agrobacterium | | Explants | Events | (Survived | TF |
| ID | strain | Construct | Plated | (Taq+) | in GH) | (%)* |
| IC-1.1 | EHA101 | 11369 | 20 | 1 | 1 | 5.0 |
| IC-1.2 | LBA4404 | 11369 | 20 | 3 | 3 | 15.0 |
| IC-1.3 | LBA4404 + | 11369 | 20 | 3 | 3 | 15.0 |
| | pSB1 | | | | | |
| MS-22 | EHA101 | 11369 | 138 | 2 | 2 | 1.44 |
| MS-23 | LBA4404 + | 11369 | 98 | 3 | 3 | 3.06 |
| | pSB1 | | | | | |
| MS-27 | LBA4404 + | 11369 | 41 | 2 | 1 | 2.43 |
| | pSB1 | | | | | |
| MS-28 | LBA4404 + | 11369 | 103 | 5 | 5 | 4.85 |
| | pSB1 | | | | | |
| MS-30 | EHA101 | 11369 | 43 | 6 | 6 | 13.95 |
| MS-37 | LBA4404 + | 11369 | 72 | 1 | 0 | 0 |
| | pSB1 | | | | | |
| MS-43 | LBA4404 + | 11369 | 108 | 1 | 1 | 0.92 |
| | pSB1 | | | | | |

*Final transformation frequency based on number of events that survived in the greenhouse

### REFERENCES

The references listed below, as well as all references cited in the specification, are incorporated herein by reference in their entireties to the extent that they supplement, explain, provide a background for, or teach methodology, techniques, and/or compositions employed herein.
Alam & Cook (1990) Anal Biochem 188:245-254.
Ballas et al. (1989) Nucleic Acids Res. 17: 7891.
Brown et al. (1987) Cell 49: 603.
Chilton (1993) Proc. Natl. Acad. Sci. USA 90: 3119.
Cubitt et al. (1995) Trends Biochem Sci 20:448-455.
DeBlock et al. (1987) EMBO J. 6: 2513.
DeBlock et al. (1989) Plant Physiol. 91: 691.
Deuschle et al. (1989) Proc. Natl. Acad. Sci. USA 86: 5400.
Deuschle et al. (1990) Science 248: 480.
Figge et al. (1988) Cell 52: 713.
Fromm et al. (1990) BioTechnology 8: 833.
Fuerst et al. (1989) Proc. Natl. Acad. Sci. USA 86: 2549.
Gordon-Kamm et al. (1990) Plant Cell 2: 603.
Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.
Guerineau et al. (1991) Mol. Gen. Genet. 262: 141.
Hooykaas (1989) Plant Mol. Biol. 13: 327.
Hu et al. (1987) Cell 48: 555.
Ishida et al. (1996) Nature Biotechnol. 14: 745.
Joshi et al. (1987) Nucleic Acids Res. 15: 9627.
Klein et al. (1987) Nature 327:70.
Komari et al. (1996) The Plant Journal 10: 165.
O. Mayo, The Theory of Plant Breeding, Second Edition (Clarendon Press, Oxford, England (1987).

Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88.
Mogen et al. (1990) Plant Cell 2:1261.
Mollony et al. (1993) Monograph Theor Appl. Genet NY 19: 148.
Munroe et al. (1990) Gene 91: 151.
Murray et al. (1989) Nucleic Acids. Res. 17: 477.
Proudfoot (1991) Cell 64: 671.
Reznikoff (1992) Mol. Microbiol. 6: 2419.
Rose & Botstein (1983) Meth Enzymol 101:167-180.
Sandford et al. (1988) Particulate Science and Technology 5:27;
Sanfacon et al. (1991) Genes Dev. 5:141.
Smith et al. (1995) Crop Science 35: 301.
Wricke and Weber, Quantitative Genetics and Selection Plant Breeding (Walter de Gruyter and Co., Berlin (1986)).
Yao et al. (1992) Cell 71: 63.
Yarranton (1992) Curr. Opin. Biotech 3: 506 (1992).
U.S. Pat. No. 5,380,831.
U.S. Pat. No. 5,436,391.
PCT International Publication No. WO 97/47763.
European Patent Application No. EP 270,356.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. An embryogenic method of transforming soybean cells or tissue, the method comprising: preparing an explant from an immature soybean cotyledon, contacting the explant with a genetic construct, and culturing the explant in the presence of a selection agent.

2. The method of claim 1, wherein preparing the explant comprises one or more of the following:
(a) isolating seeds from seed pods and sterilizing the seeds.
(b) removing the seed coat;
(c) isolating the immature cotyledons from the explant;
(d) pre-culturing the immature cotyledons; and
(e) wounding the explant.

3. The method of claim 2, wherein the wounding comprises one of the following:
(i) contacting a mechanical instrument with the abaxial or adaxial sides of the cotyledons; and
(ii) bombarding the explant tissue with a microprojectile on the abaxial or adaxial sides of the cotyledons.

4. The method of claim 1, wherein the genetic construct comprises a gene of interest and/or a selectable marker gene.

5. The method of claim 4, wherein the selectable marker gene confers mannose resistance to the explant.

6. The method of claim 4, wherein the selectable marker gene confers antibiotic or herbicide resistance to the explant.

7. The method of claim 6, wherein the antibiotic is selected from the group consisting of: hygromycin, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, vancomycin, and combinations thereof.

8. The method of claim 6, wherein the herbicide is selected from the group consisting of: glyphosate, sulfonylurea, imidazolinone, glufosinate, bialophos, phenoxy proprionic acid, cycloshexone, triazine, benzonitrile, HPPD inhibitors and combinations thereof.

9. The method of claim 1, wherein the contacting comprises contacting the explant with an Agrobacterium cell comprising the genetic construct.

10. The method of claim 9, wherein the Agrobacterium is Agrobacterium tumefaciens.

11. The method of claim 9, wherein the explant is contacted with the Agrobacterium for about 45 minutes.

12. The method of claim 11, wherein the explants are further co-cultured with Agrobacterium for up to about 5 days.

13. The method of claim 1, wherein the contacting comprises delivering the genetic construct to the explant using a ballistic device.

14. The method of claim 1, further comprising culturing the explant on a culture medium comprising a plant hormone.

15. The method of claim 2, wherein the pre-culturing is for up to about 5 days.

16. The method of claim 2, wherein the contacting comprises abrading, piercing, poking, penetrating with fine particles or pressurized fluids, plasma wounding, applying of hyperbaric pressure, sonicating, or combinations thereof.

17. The method of claim 2, wherein the mechanical instrument comprises a scalpel, pair of scissors, needle, abrasive object, airbrush, particle, vacuum infiltration, electric gene gun, sound wave, or combinations thereof.

18. The method of claim 2, wherein the microprojectile comprises metal, glass, silica, ice, polyethylene, polypropylene, polycarbonate, carbon compounds, and combinations thereof.

19. The method of claim 18, wherein the metal is gold.

20. A transgenic soybean cell or tissue prepared according to the method of claim 1.

21. A soybean plant regenerated from the transgenic soybean cell or tissue of claim 20.

22. A transgenic seed produced by the soybean plant of claim 21.
